(19) United States
(12) Patent Application Publication
(10) Pub. No.: US 2026/0345478 A1
(43) Pub. Date: Nov. 5, 2026

(54) MANGO FRUIT EXTRACT COMPOSITIONS AND METHODS THEREOF FOR SKIN CARE

(71) Applicants: ANJU MAJEED, Bangalore (IN); SHAHEEN MAJEED, Payson, UT (US); MOHAMMAD MANSOOR ANSARI, Bangalore (IN); LAKSHMI MUNDKUR, Bangalore (IN); SYED MUSTHAFA KHADAR, Bangalore (IN)

(72) Inventors: ANJU MAJEED, Bangalore (IN); SHAHEEN MAJEED, Payson, UT (US); MOHAMMAD MANSOOR ANSARI, Bangalore (IN); LAKSHMI MUNDKUR, Bangalore (IN); SYED MUSTHAFA KHADAR, Bangalore (IN)

(21) Appl. No.: 19/488,064

(22) PCT Filed: May 27, 2024
(86) PCT No.: PCT/US2024/031194

§ 371 Date: Nov. 25, 2025

(30) Foreign Application Priority Data
May 27, 2023 (IN) 202341036706

(51) Int. Cl. A61K 8/9794; A61K 9/16; A61K 31/192; A61K 31/352; A61K 31/366; A61K 31/7048; A61K 36/22; A61P 37/08; A61P 39/06; A61Q 19/02; A61Q 19/08

(52) U.S. Cl. CPC A61K 8/9794; A61K 9/1652; A61K 9/1694; A61K 31/192; A61K 31/352; A61K 31/366; A61K 31/7048; A61K 36/22; A61P 37/08; A61P 39/06; A61Q 19/02; A61Q 19/08; A61K 2236/333; A61K 2236/39; A61K 2236/51; A61K 2236/53; A61K 2236/55

(57) ABSTRACT

The present invention discloses a standardised polyphenol composition from Mangifera indica. Further, the invention discloses the use of the abovementioned composition for skin care by reducing signs of skin ageing, rejuvenating the skin as an antioxidant, as a skin lightening agent and providing an even skin tone.

### CROSS REFERENCE TO RELATED APPLICATIONS

This application is a US national stage application of PCT application no. PCT/US2024/031194 filed on 27 May 2024, claiming priority from Indian Provisional application no. IN 202341036706, filed on 27 May 2023, the contents of which is incorporated herein.

### FIELD OF INVENTION

The invention is directed towards a composition comprising Mangifera indica extract. More specifically, the invention is directed to a polyphenol composition from Mangifera indica extract and its use in management of skin ageing.

### BACKGROUND OF THE INVENTION

Mangifera indica is a well-known tropical fruit in the world with a history of thousands of years in ayurvedic medicine. Every aspect of mango, either ripe or raw, such as leaves, bark, seed, root, or kernel have pharmacological properties [See: Ediriweera et al., A review on Ethnopharmacological Applications, Pharmacological Activities, and Bioactive Compounds of Mangifera indica (Mango), Evidence-Based Complementary and Alternative Medicine (2017)]. Nutritional value of mango fruit is dependent on the phytoconstituents, which vary depending on the time of harvesting, origin and geographical conditions. It is imperative that understanding the changes in the chemical composition in mango fruit during its development will allow producers to better characterize their cultivars and select those that have phytochemical characteristics that give added value to the fruit. One of the best ways to understand and reap the benefits of Mango is through standardization of Phytoactives to optimize for diverse geographical origins of the fruit.

The human skin is exposed to a wide range of environmental pollutants and harmful UV radiation, damaging the skin and leading to premature aging. Exposure to these harmful pollutants and UV radiation will increase the production of reactive oxygen species (ROS) and inflammatory markers, increasing the oxidative stress and inflammation in the skin cells thereby damaging the cellular biomolecules (lipids, sugars, proteins, and polynucleotides). Protection against these agents will eventually improve the skin texture, which leads to graceful aging. The skin cells inherently possess cellular defense systems against ROS and inflammatory agents, which include nonenzymatic molecules (glutathione, vitamins A, C, and E, and several antioxidants present in foods) and enzymatic scavengers of ROS, with superoxide dismutase (SOD), catalase (CAT), and glutathione peroxidase (GPX). Apart from these, many natural molecules confer protection against the UV and ROS induced damage which constitutes the focus of cosmetic industry.

Botanical extracts have been reported to exhibit skin lightening and anti-ageing effects [See: Zhu and Gao, The Use of Botanical Extracts as Topical Skin-Lightening Agents for the Improvement of Skin Pigmentation Disorders, Journal of Investigative Dermatology Symposium Proceedings 13 (2008) Pgs. 20-24]. Japanese patent JPH0782134, also discloses a cosmetic composition for preventing damage due to ultraviolet rays comprising L-3-O-ethyl-ascorbic acid and natural molecule kojic acid. Curcuminoids from Curcuma longa is known to inhibit tyrosinase enzyme and melanin production (Tu et al., Curcumin inhibits melanogenesis in human melanocytes, Phytother Res. 2012; 26(2): 174-9). Continuing efforts to find a safe and effective natural ingredient for complete skin care including skin lightening and anti-ageing effects are undertaken, and this invention provides one such effective composition comprising polyphenols from Mangifera indica. U.S. Pat. No. 10,646,527 B2 disclose compositions comprising polyphenols from Mangifera indica for the treatment of obesity, wherein the extract was prepared from leaf, bark, and skin is reported in literature. However, the skin care effects of polyphenols from Mangifera indica is not well documented.

The present invention solves the problems by standardizing Phytoactives in mango fruit for applications in skin care.

### OBJECTIVES OF THE INVENTION

It is a general objective of the invention to disclose a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid.

It is another objective of the invention, to disclose the potential of a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid in the management of skin ageing, skin rejuvenation and skin brightening.

### SUMMARY

The first aspect of the invention relates to a standardised polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid.

The second aspect of the invention covers a method of managing skin ageing in a subject, comprising a) identifying the subject showing signs of skin ageing b) administering effective dose of a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid; to bring about the effects of reduction in skin aging, skin rejuvenation, anti-allergic and skin brightening properties.

Yet another aspect, the invention covers use of a composition comprising standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid as a skin care agent in a subject.

In yet another aspect of the invention covers a composition comprising standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid for use as skin care agent in a subject

The broader scope of applicability of the present invention will be apparent from the detailed description below. However, it should be understood that the detailed description and specific examples, while indicating preferred embodiments of the invention, should not be construed as the limitations to the invention, and it is within the scope of those skilled in the art to make various changes and modifications, such as changing the concentration range of composition used, derivatives/analogs of composition used herewith, experimental conditions, choice of mammals, to the spirit and scope of the invention from this detailed description.

### BRIEF DESCRIPTION OF DRAWINGS

FIGS. 1A and 1B are graphical representations of dose response suggesting inhibition of DPPH and ROS scavenging activity using the standardized polyphenol composition from Mangifera indica.

FIGS. 2A and 2B are graphical representations of a dose response suggesting inhibition of histamine release (FIG. 2A) and hexosaminidase release (FIG. 2B) using the standardized polyphenol composition from Mangifera indica

FIGS. 3A and 3B are graphical representations of a dose response suggesting Tyrosinase inhibition (FIG. 3A) and Melanin inhibition (FIG. 3B) using the standardized polyphenol composition from Mangifera indica

FIGS. 4A, 4B, 4C, 4D, and 4E are graphical representations of a dose response suggesting inhibition of Hyaluronidase (FIG. 4A), Collagenase (FIG. 4B), Elastase (FIG. 4C), Vesperlysine-like AGE formation (FIG. 4D), and pentosidine like AGE formation (FIG. 4E) using the standardized polyphenol composition from Mangifera indica.

### DESCRIPTION OF PREFERRED EMBODIMENTS

### Selected Definitions

All the terms used in this application carry ordinary meaning as known in the prior art unless otherwise specified. Few other specific definitions used in this invention are explained below, which applies throughout this specification. Claims provide broader definition unless and otherwise specified.

Furthermore, the terms “approximately,” “approximate,” “about,” and similar terms generally refer to ranges that include the identified value within a margin of 20%, 10%, or preferably 5%, and any values there between.

As used herein, treatment or management of a condition refers to effectively ameliorating conditions disclosed in the invention.

As used herein, IC₅₀ refers to half maximal inhibitory concentration, which is a measure of how much of a particular inhibitory substance (e.g. drug) is needed to inhibit, in vitro, a given biological process or biological component by 50%.

As used herein, Mangiophelin® refers to a brand which is standardized to contain not less than 10% polyphenols (10-60% w/w), wherein the polyphenols comprises of 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid

As used herein, % w/w refers to purity of the component.

In the most preferred embodiment, the invention discloses a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid. In a related embodiment of the invention, the composition is isolated from Mangifera indica fruit using a process comprising:
a) Charging coarse powder of immature deseeded dry fruit in a cleaned reactor.
b) Charging 3 volumes of Ethanol to the same reactor and stir at 60-65 °C for three hours.
c) Filtering the mass from step b, through nutsche filter.
d) Repeating steps b and c twice.
e) Combining Ethanol filtrates and concentrating to reduce the volume to ⅛th of the original volume.
f) Washing twice with equal volumes of Ethanol by stirring 1 hr each time at 50 °C and strip off Ethanol (to remove moisture from the concentrate);
g) Dividing the same (Ethanol concentrate) into equal fractions (fraction 1 and fraction 2).
h) Concentrating fraction 1 from step g to pourable mass and dry the product completely at 55-60 °C under high vacuum in vacuum tray dryer (VTD) to yield hygroscopic powder and assayed for polyphenols by UV spectrophotometry.
i) Dissolving fraction 2 from step g with two volumes of de-mineralized water and partition three times with equal volumes of n-Heptane.
j) Partitioning the aqueous phase from step i three times with equal volumes of ethyl acetate.
k) Concentrating the ethyl acetate portion to pourable mass at 60-65 °C under high vacuum.
l) Drying the product completely at 55-60 °C under high vacuum in VTD to yield hygroscopic powder and assayed for polyphenols by UV.
m) In the left-over aqueous layer from step j, add Maltodextrin (60%) and spray dry.
n) Blending the final product in the ratio of 55.55% initial extract powder (obtained from step h) and 9.02% of enriched extract of step 1 with 35.42% of spray dried material. The product obtained was assayed for polyphenols by UV.
Wherein the polyphenols content is 14% w/w from step h and 60% w/w from step 1 and 12% w/w from step n.
It is to be noted that the present Mangifera indica extraction process involves the solvents—Ethanol, Heptane and Ethyl acetate, however, it is within the skill of the person skilled in the art to use any other solvents selected from, but not limited to methanol, hexane, butanol, Acetic acid, Acetone, Isobutyl acetate, Anisole, Isopropyl acetate, Methyl acetate, 3-Methyl-1-butanol, Butyl acetate, Methylethyl ketone, tert-Butylmethyl ether, 2-Methyl-1-propanol, Pentane, Pentanol, Propanol to arrive at a similar composition.

In another most preferred embodiment of the invention, the invention discloses use of a composition comprising a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid as a skin care agent in a subject. In a related embodiment, the composition confers skin protection by reducing signs of skin aging, rejuvenating the skin, reducing allergy, and brightening skin tone. In a related embodiment of the invention, wherein polyphenols are in the range 10-60% w/w. In another related embodiment of the invention, wherein the composition reduces skin aging through collagen enhancement by inhibiting collagenase enzyme, anti-glycation activity, and anti-elastase activity. In yet another related embodiment of the invention, the composition rejuvenates skin by being an anti-oxidant and inhibiting free radicals. In yet another related embodiment of the invention, the composition confers anti-allergic activity by inhibiting Histamine and β-hexosaminidase release from mast cells. In another embodiment of the invention, composition confers skin brightening effect by inhibiting Melanin production and Tyrosinase activity. In another related embodiment, the signs of skin aging include Wrinkles, Xeroderma, Age spots, Loss of elasticity, Rough skin, Sagging skin, Uneven skin tone, dullness and presence of visible pores and skin tag. In another embodiment of the invention, wherein the composition further comprises of stabilizing agents, bioavailability enhancers, antioxidants, cosmeceutically or nutraceutically accepted excipients, and administered orally or topically in the form of creams, lotions, ointments, serum, emulsions. In another embodiment of the invention, wherein the subject is a mammal.

In another most preferred embodiment of the invention, the invention discloses a composition comprising a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid for use as a skin care agent in a subject. In a related embodiment, the composition confers skin protection by reducing signs of skin aging, rejuvenating the skin, reducing allergy, and brightening skin tone. In a related embodiment of the invention, wherein polyphenols are in the range 10-60% w/w. In another related embodiment of the invention, wherein the composition reduces skin aging through collagen enhancement by inhibiting collagenase enzyme, anti-glycation activity, and anti-elastase activity. In yet another related embodiment of the invention, wherein the composition rejuvenates skin by being an anti-oxidant and inhibiting free radicals. In yet another related embodiment of the invention, the composition confers anti-allergic activity by inhibiting Histamine and β-hexosaminidase release from mast cells. In another embodiment of the invention, composition confers skin brightening effect by inhibiting Melanin production and Tyrosinase activity. In another related embodiment, the signs of skin aging include Wrinkles, Xeroderma, Age spots, Loss of elasticity, Rough skin, Sagging skin, Uneven skin tone, dullness and presence of visible pores and skin tag. In another embodiment of the invention, wherein the composition further comprises of stabilizing agents, bioavailability enhancers, antioxidants, cosmeceutically or nutraceutically accepted excipients, and administered topically in the form of creams, lotions, ointments, serum, emulsions. In another embodiment of the invention, wherein the subject is a mammal. In a related embodiment of the invention, the composition can also be applied orally.

In another most preferred embodiment of the invention, the invention discloses a method of managing skin ageing in a subject, comprising a) identifying the subject showing signs of skin ageing b) administering effective dose of a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid; to bring about the effects of reduction in skin aging, skin rejuvenation, anti-allergic and skin brightening properties.

In a related embodiment of the invention, wherein polyphenols are in the range 10-60% w/w. In another related embodiment of the invention, wherein the composition reduces skin aging through collagen enhancement by inhibiting collagenase enzyme, anti-glycation activity, and anti-elastase activity. In yet another related embodiment of the invention, wherein the composition rejuvenates skin by being an antioxidant and inhibiting free radicals. In yet another related embodiment of the invention, the composition confers anti-allergic activity by inhibiting Histamine and β-hexosaminidase release from mast cells. In another embodiment of the invention, composition confers skin brightening effect by inhibiting Melanin production and Tyrosinase activity. In another related embodiment, the signs of skin aging include Wrinkles, Xeroderma, Age spots, Loss of elasticity, Rough skin, Sagging skin, Uneven skin tone, dullness and presence of visible pores and skin tag. In another embodiment of the invention, wherein the composition further comprises of stabilizing agents, bioavailability enhancers, antioxidants, cosmeceutically or nutraceutically accepted excipients, and administered topically in the form of creams, lotions, ointments, serum, emulsions. In another embodiment of the invention, wherein the subject is a mammal.

In all the related embodiment of the invention, the polyphenols include, but not limited to Cyanidin, Catechin, Kaempferol, Myricetin, Proanthocyanidin dimers, Proanthocyanidin trimers, Gallic acid, Ellagic acid, Coumarin, Caffeic acid, Vanillin, Cinnamic acid, Ferulic acid, Mangiferin, Mangiferin gallate, Isomangiferin, Isomangiferin gallate, Quercetin, Rhamnetin 3-0-galactoside/glucoside, Gallotannins, β-glucogallin, Rhamnetin, Quercetin 3-0-galactoside. Similarly, flavonoids can be standardized to Catechins, Quercetin, Kaempferol, Rhamnetin, Mangiferin, Glycosides of quercetin (glucose, galactose, rhamnose, xylose, and arabinose), Isorhamnetin, Fisetin, Myricetin, Apigenin, Luteolin

### EXAMPLES

The examples are provided solely for illustration and are not to be construed as limitations of the present disclosure, as many variations thereof are possible without departing from the spirit and scope of the present disclosure.

### Example 1: Materials

As used herein, Mangiophelin® refer to standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid

As used herein, Mangiophelin® is prepared by a process from Mangifera indica fruit comprising:
a) Charging 500 kg of coarse powder of immature deseeded dry fruit in a cleaned reactor;
b) Charging 3 volumes of Ethanol (1500 L) to the same reactor and stir at 60-65 °C for three hours;
c) Filtering the mass from step b, through nutsche filter;
d) Repeating the steps b and c two more times with 1500 L and 1000 L of Ethanol;
e) Combining Ethanol filtrates, and concentrate to reduce the volume to 500 L;
f) Washing two more times with 500 L of Fresh Ethanol by stirring 1 hr each time at 50 °C and strip off Ethanol (to remove moisture from the concentrate);
g) Reducing final volume to 350 L and divide into 175 L each;
h) Concentrate 175 L portion from step g to pourable mass and dry the product completely at 55-60 °C under high vacuum in vacuum tray dryer (VTD) to yield 100 kg of hygroscopic powder and assayed for polyphenols by UV;
i) Dissolve remaining 175 L of Ethanol concentrate from step g with two volumes of de-mineralized water and partition three times with n-Heptane (100 L×3 times);
j) Partition the aqueous phase from step i three times with ethyl acetate (100 L×3 times);
k) Concentrate the ethyl acetate portion to pourable mass at 60-65 °C under high vacuum;
l) Dry the product completely at 55-60 °C under high vacuum in VTD to yield 16.25 kg of hygroscopic powder and assayed for polyphenols by UV spectrophotometry;
m) In the left-over aqueous layer (approx. 74 kg) from step j add 106 kg Maltodextrin and spray dry.
n) Blend 100 kg of initial extract powder (obtained from step h) and 16.25 kg of enriched extract of step 1 with 63.75 kg of spray dried material to yield 180 kg of powder and assayed for polyphenols by UV spectrophotometry.
Wherein the polyphenols content is 14% w/w from step h and 60% w/w from step 1 and 12% w/w from step n (Table 1).

TABLE 1
Total polyphenol content from different
batches of Mangiophelin ®

| | Polyphenols | |
|---|---|---|
| Batch No | Content by UV | |
| Batch 1 | 23.0% | w/w |
| Batch 2 | 61.1% | w/w |
| Batch 3 | 13.3% | w/w |
| Batch 4 | 18.0% | w/w |
| Batch 5 | 18.0% | w/w |
| Batch 6 | 18.0% | w/w |
| Batch 7 | 12.5% | w/w |
| Batch 8 | 10% | w/w |

The presence of individual polyphenols were detected using analytical HPLC using known methods in the state of art using reference standards and the composition was standardized to 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid.

### Example 2: Mangiophelin® for Skin Rejuvenation

The skin is exposed to various external environmental pollutants leading to the formation of free radicals which damage the skin and accelerate skin ageing. Antioxidants can delay or inhibit the process of oxidation of cellular molecules, quench the free radicals formed, or that can upregulate antioxidant defense systems [See: Jaganjac et al. Short Overview of Some Assays for the Measurement of Antioxidant Activity of Natural Products and Their Relevance in Dermatology, Molecules 26 (17) 2021, Pgs. 5301] and protect the skin against damage and rejuvenate the skin. The Antioxidant and skin rejuvenation potential of Mangiophelin® was established using different in-vitro assays.

### In-Vitro Antioxidant Activity—DPPH Radical Scavenging Activity

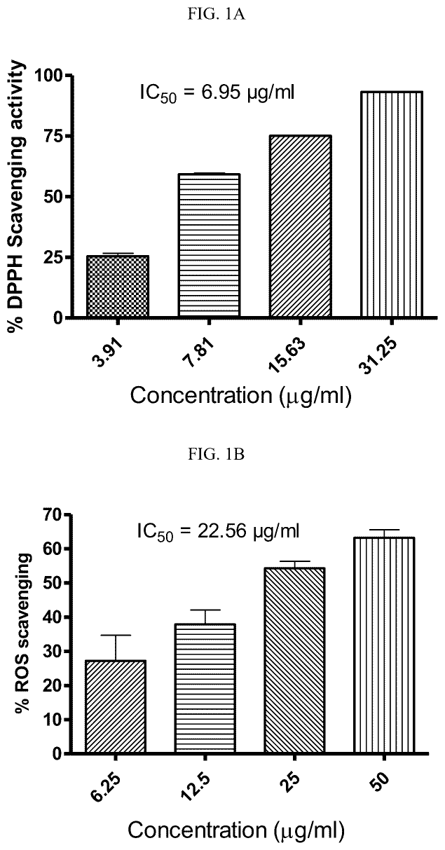

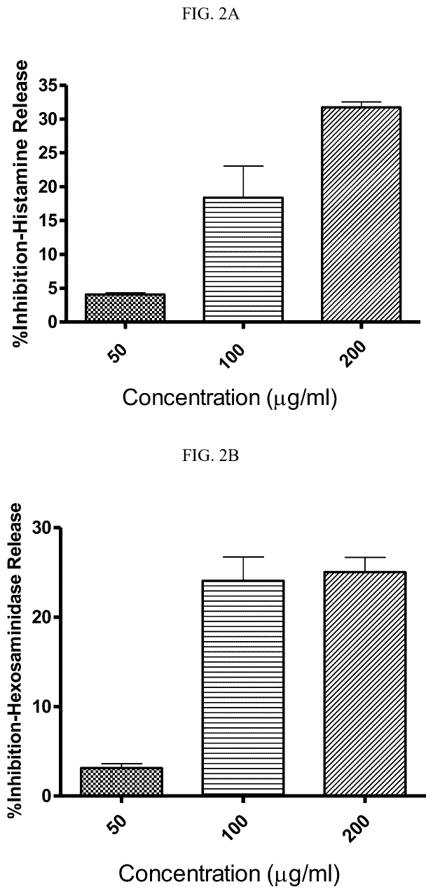

The antioxidant potential of Mangiophelin® was estimated based on the scavenging activity of stable 1,1-diphenyl-2-picrylhydrazyl (DPPH) free radical. DPPH is a stable free radical wherein the free electron is delocalized over the entire molecule unlike the other free radicals, which dimerize. This delocalization gives its deep violet color, characterized by an absorption band in ethanol solution centered at about 517 nm. When a solution of DPPH is mixed with that of a substrate (AH) that can donate a hydrogen atom, then this gives rise to the reduced form with the loss of this violet color. Briefly, 20 μl of various concentrations of Mangiophelin® was mixed with 180 μl of Ethanolic solution of DPPH (0.066 mM) in a 96-well microplate. The plates were incubated in the dark at room temperature for 30 min and the absorbance was measured at 517 nm (Table 1, FIG. 1) using a Tecan Infinite M Nano microplate reader [See: Herald et al. High-throughput micro plate assays for screening flavonoid content and DPPH-scavenging activity in sorghum bran and flour, J. Sci Food Agric. 92 (2012) Pgs. 2326-2331].

The percentage of the DPPH radical scavenging is calculated using the equation as given below:

% inhibition of DPPH radical=([A_{br}−Aₐᵣ]/A_{br})×100

where A_{br} is the absorbance before reaction and Aₐᵣ is the absorbance after reaction has taken place. A dose dependent DPPH scavenging activity is shown in FIG. 1A, with IC₅₀ value of 6.95 μg/ml (Table 2).

TABLE 2
DPPH Scavenging Assay

| | |
|---|---|
| | % DPPH scavenging |
| Mangiophelin ® | activity |

| Conc. (μg/ml) | Mean | SD |
|---|---|---|
| 3.91 | 25.42 | 1.30 |
| 7.81 | 59.18 | 0.58 |
| 15.63 | 75.15 | 0.20 |
| 31.25 | 93.21 | 0.09 |

IC₅₀ = 6.95 μg/ml

In-Vitro Antioxidant Activity—H₂O₂ Induced ROS Scavenging

Hydrogen peroxide (H₂O₂) is an endogenous reactive oxygen species that contributes to oxidative stress directly as a molecular oxidant and indirectly through free radical generation. The DNA damage in the body is caused by lipid peroxidation initiated by hydroxy radical, which was decomposed from H₂O₂. Swiss 3T3 fibroblast cells (3×10⁴ cells/well) were seeded in 96-well black microplates and allowed to grow as a monolayer for 24 h in DMEM media with 10% FBS. Cells were induced with H₂O₂ (20 mM) in DMEM media with 2% FBS with or without different concentrations of Mangiophelin® for three hours. Freshly prepared Dichloro-dihydro-fluorescein diacetate (DCFH-DA) reagent was added to all the wells at a concentration of 10 μg/well and incubated at 37 °C for 30 min. The fluorescence was recorded at a wavelength of 485:520 (Ex: Em) nm in BMG FluoStar Optima [See: LeBel, et al. Evaluation of the probe 2′,7′-dichlorofluorescin as an indicator of reactive oxygen species formation and oxidative stress. Chem. Res. Toxicol. 5 (1992), Pgs. 227-231]. A dose-dependent ROS-scavenging activity of Mangiophelin® was observed (FIG. 1B).

TABLE 3
H₂O₂ induced ROS Scavenging Assay

| | |
|---|---|
| | % ROS scavenging |
| Mangiophelin ® | activity |

| Conc. (μg/ml) | Mean | SD |
|---|---|---|
| 6.25 | 27.23 | 7.47 |
| 12.5 | 37.87 | 4.23 |
| 25 | 54.27 | 2.04 |
| 50 | 63.23 | 2.33 |

IC50 = 22.56 μg/ml

### In-Vitro Antioxidant Activity—Oxygen Radical Absorbance Capacity (ORAC) Method

This assay is based on generation of free radical using AAPH (2,2-azobis 2-amidopropane dihydrochloride) and measurement of decrease in fluorescence in the presence of free radical scavengers. Different concentrations of Mangiophelin® were pipetted into each well (25 μl/well) followed by 150 μl of AAPH reagent (2,2′-Azobis(2-amidinopropane) dihydrochloride; final conc. 10×10⁻²M) in 75 mM potassium phosphate buffer (pH 7.4). Then 150 μl of disodium fluorescein dye (final conc. 4.8×10⁻⁷M) was added and mixed before the initial reading (f₀) was taken. Fluorescence readings were taken (BMG Fluostar Optima Microplate Reader) at 485/520 nm after every 1 minute for 35 minutes (f₁ . . . f₃₅). Trolox standard from 12.5-200 μM was kept as standard. The ORAC activity was expressed in terms of μmol Trolox equivalence per gram sample (μmol TE/g) [See: Huang, D., Ou, B., Hampsch-Woodill, M., Flanagan, J. A., & Prior, R. L. (2002). High-throughput assay of oxygen radical absorbance capacity (ORAC) using a multichannel liquid handling system coupled with a microplate fluorescence reader in 96-well format. Journal of agricultural and food chemistry, 50(16), 4437-4444].

### Example 3: Mangiophelin® as Anti-Allergy Agent

An allergic reaction is characterized by the hypersensitivity of the human immune system towards foreign substances. The initial phase of allergic reaction occurs within minutes of exposure to allergens. It is the crosslink between the allergen and high-affinity receptor for immunoglobin E complex triggers the degranulation of the mast cell which leads to the release of chemokines, cytokines, and the release of lipid-derived mediators and vasoactive amine [See: Rani et al. Mechanistic Studies of the Antiallergic Activity of Phyllanthus amarus Schum. & Thonn. And Its Compounds Molecules 26(3) 2021 Pg. 695]. Various external allergens can trigger allergic reactions in the skin and cause damage and increase signs of ageing. Mangiophelin® was evaluated for its anti-allergic property by its potential to inhibit histamine and β-hexosaminidase. P815 cell line (Mouse, mast cells) were procured from National Center for Cell Sciences (NCCS), Pune, India, and were grown in DMEM media supplemented with 10% FBS in 5% CO₂ humidified incubator.

### Anti-Allergy Study-Histamine Assay

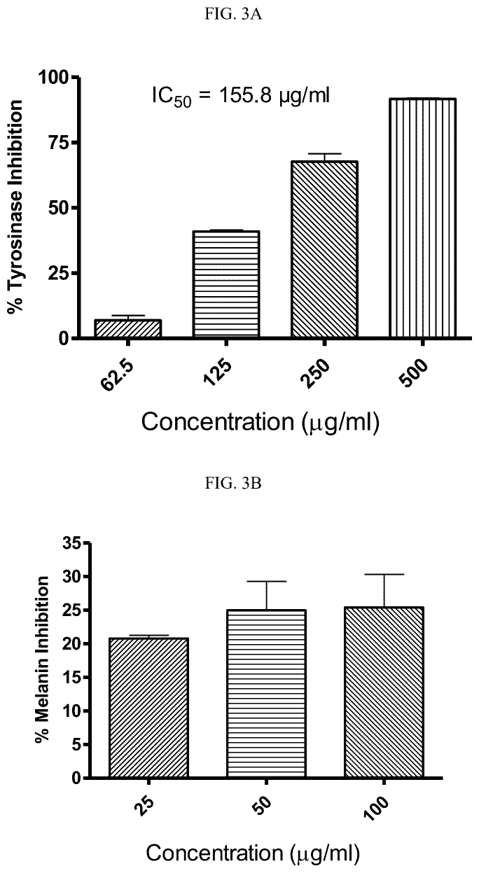

5×10⁵ P815 cells were induced with 40 μg/ml of C48/80 (a polymer from the condensation of N-methyl-p-methoxy phenethylamine with formaldehyde), which causes mast cell degranulation and a promoter of Histamine release, along with different concentrations of Mangiophelin® and incubated for a period of 90 mins. Reaction was terminated by placing the cells on ice for 10 minutes and centrifuged for 10 minutes at 1200 rpm. 100 μl of cell supernatant was mixed with 0.05 incubated in the presence of NaOH (2N), and ortho-phthalaldehyde (OPT) (0.625 mg/ml) for exactly 10 min. The reaction was stopped by the addition of 50 μl of HCl (IN), and the fluorescence was measured on BMG Fluostar Optima microplate reader at Ex/Em: 320/460 nm (Table 4) [See: Andersson, M., et al., Measurement of histamine in nasal lavage fluid: comparison of a glass fiber-based fluorometric method with two radioimmunoassays. J Allergy Clin Immunol, 1990. 86(5): pgs. 815-20]. A dose-dependent inhibition of histamine release observed (FIG. 2A).

TABLE 4
Inhibition of Histamine Release using Mangiophelin ®

| | |
|---|---|
| | % inhibition in |
| Mangiophelin ® | histamine release |

| Conc. (μg/ml) | Mean | SD |
|---|---|---|
| 50 | 4.08 | 0.23 |
| 100 | 18.40 | 4.66 |
| 200 | 31.71 | 0.82 |

### Anti-Allergy Study: β-Hexosaminidase Assay

5×10⁵ P815 cells were induced with 40 μg/ml of C48/80 along with different concentrations of Mangiophelin® and incubated for a period of 90 mins. Reaction was terminated by placing the cells on ice for 10 minutes and centrifuged for 10 minutes at 1200 rpm. To the 50 μl of supernatant, 100 μl p-nitrophenyl-N-acetyl-β-D-glucosamine (PNAG) solution was added and incubated at 37 °C for 90 min. After incubation, 50 μl of 0.4 M Glycine was added to each well. The absorbance was measured at 405 nM with reference filter at 620 nm using Tecan Infinite M Nano microplate reader [See: Kuehn H S, Radinger M, Gilfillan A M. Measuring mast cell mediator release. Curr Protoc Immunol. 2010 November, Chapter 7]. A dose dependent inhibition of the release of β-Hexosaminidase was observed (FIG. 2B)

TABLE 5
Inhibition of β-Hexosaminidase release using Mangiophelin ®

| | |
|---|---|
| | % inhibition in |
| Mangiophelin ® | hexosaminidase release |

| Conc. (μg/ml) | Mean | SD |
|---|---|---|
| 50 | 3.13 | 0.48 |
| 100 | 24.06 | 2.64 |
| 200 | 25.03 | 1.63 |

### Example 4: Mangiophelin® for Skin Lightening

Melanin is a pigment in mammalian skin to protect the skin against harmful effects of UV irradiation. However, abnormal accumulation results in skin issues such as lentigo, age spots, and melisma. Melanin is synthesized via a series of enzymatic reactions called melanogenesis. In effect, inhibitors of melanogenesis could lead to the identification of effective skin-whitening compounds for medicines and cosmetics. Tyrosinase is one of the key enzymes involved in melanogenesis [See: Lee et al. Tyrosinase-Targeting Gallacetophenone Inhibits Melanogenesis in Melanocytes and Human Skin-Equivalents 21(9) 2020 3144].

### Mangiophelin® for Skin Lightening-Anti-Tyrosinase Activity

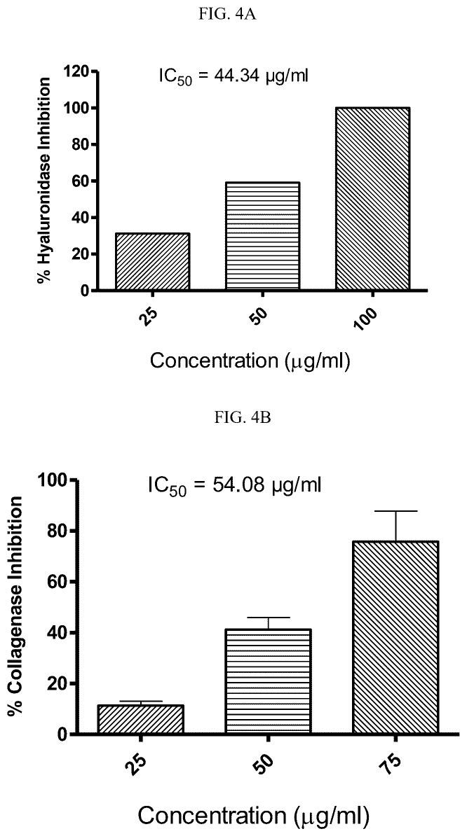

To understand the inhibitory potential in Tyrosinase, an assay was performed in a 96 well clear microtiter plate. Varying concentrations of Mangiophelin® in DMSO or (Phosphate buffer (PBS)) were pre-incubated with 30 units of Mushroom Tyrosinase enzyme at 37 °C for 10 minutes. The reaction was initiated by adding 2.5 mM L-Tyrosine disodium and the absorbance was read immediately every minute until 15 minutes using the microplate reader (Tecan Infinite M Nano) at 492 nm wavelength [See: Jeong et al. Tyrosinase inhibitor isolated from the leaves of Zanthoxylum piperitum. Bioscience, biotechnology, and biochemistry, 68(9), 2004, Pgs. 1984-1987}]. A dose inhibition of tyrosinase was observed (FIG. 3A, Table 6)

TABLE 6
Inhibition of Anti-Tyrosinase Activity by Mangiophelin ®

| | |
|---|---|
| | % Tyrosinase enzyme |
| Mangiophelin ® | inhibition |

| Conc. (μg/ml) | Mean | SD |
|---|---|---|
| 62.5 | 6.94 | 1.89 |
| 125 | 40.92 | 0.65 |
| 250 | 67.69 | 2.98 |
| 500 | 91.67 | 0.39 |

IC50 = 155.8 μg/ml

### Mangiophelin® for Skin Lightening-Melanin Inhibition

B16-F1 mouse melanoma cells are cultured for 24 hours in a 96 well microtiter plate at a seeding density of 5000 cells per well. The cells were treated with varying non-cytotoxic concentrations of the Mangiophelin® over a period of 3 days in DMEM media with 10% FBS in the presence of α-MSH (50 nM). The melanin is extracted by IN NaOH and the absorbance was read at 490 nm wavelength in a microplate reader (Tecan Infinite M Nano) [See: Choi et al. (4-Methoxy-benzylidene)-(3-methoxy-phenyl)-amine, a nitrogen analog of stilbene as a potent inhibitor of melanin production. Chemical and pharmaceutical bulletin, 50(4), 2002, Pgs. 450-452]. The inhibitory effect of the sample was calculated based on the degree of inhibition of melanin formation when compared to the untreated control wells (Table 7, FIG. 3B).

TABLE 7
Melanin Inhibition by Mangiophelin ®

| | |
|---|---|
| Mangiophelin ® | % Melanin inhibition |

| Conc. (μg/ml) | Mean | SD |
|---|---|---|
| 25 | 20.78 | 0.46 |
| 50 | 24.98 | 4.30 |
| 100 | 25.40 | 4.93 |

### Example 5: Mangiophelin® as Anti-Ageing Agent

Ageing of the skin depends on a variety of factors which include lifestyle, diet, heredity, and other personal habits. Exposure to external stimuli and environmental pollutants can produce free radicals, and damage cells, leading to premature wrinkles, other factors that contribute to aging of the skin include stress, sun exposure, and obesity. The potential of Mangiophelin® in preventing skin ageing was evaluated using different experimental assays.

### Anti Hyaluronidase Activity

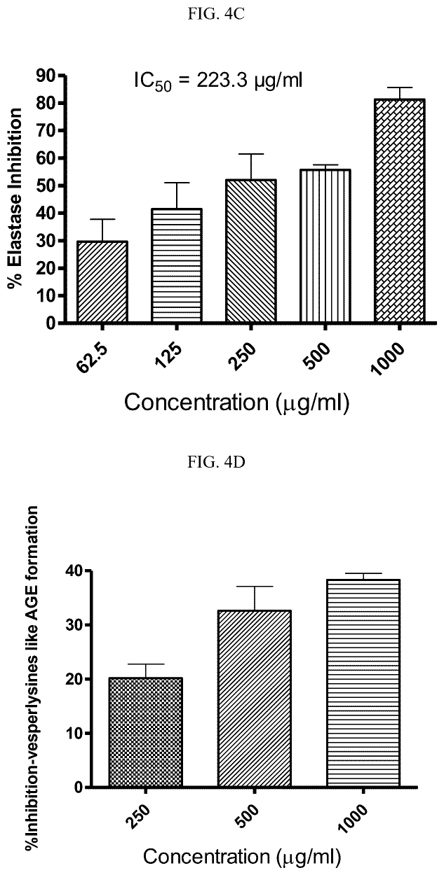

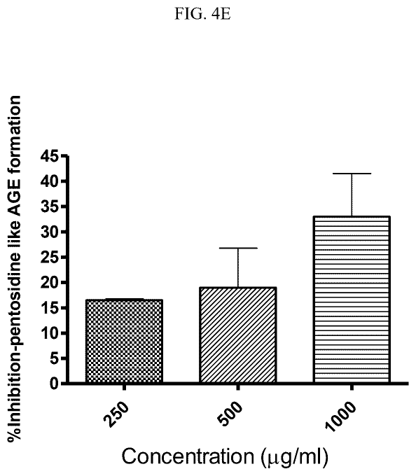

Hyaluronic acid or hyaluronan (HA), a glucose-based polymer, is commonly found in tissues and fluids of the body, but it is abundantly found in the dermal compartment of skin and the epidermal layer. HA mainly promotes skin rejuvenation, contains moisture, increases viscosity, and reduces the permeability of extracellular fluid. Hyaluronidase is an HA-destructive enzyme which leads to loss of strength, flexibility and moisture, and subsequently, skin aging. A volume of 25 μl hyaluronidase (30 U/ml) in enzyme diluent (20 mM Sodium Phosphate buffer with 77 mM Sodium Chloride and 0.01% (w/v) Bovine Serum Albumin, pH 7.0 at 37 °C) were mixed with 25 μl of different concentrations of sample in enzyme diluent and incubated at 37 °C for 10 min. The reaction was then initiated by the addition of 50 μl of Mangiophelin® in the form of hyaluronic acid solution (0.05% HA in 300 mM sodium phosphate, pH 5.35 at 37 °C) and incubated at 37 °C for 45 min. The undigested hyaluronic acid was precipitated with 200 μl of acidic albumin solution (24 mM Sodium Acetate, 79 mM Acetic Acid with 0.1% (w/v) BSA, pH 3.75). The absorbance of the reaction mixture was measured at 600 nm using a microplate reader (Tecan Infinite M Nano) [See: Razak et al. “Assessment of cosmeceutical potentials of selected mushroom fruitbody extracts through evaluation of antioxidant, anti-hyaluronidase and anti-tyrosinase activity.” J 3(3) 2020, Pgs. 329-342]. A dose-dependent inhibition of Hyaluronidase activity was observed (Table 8, FIG. 4A).

TABLE 8
Hyaluronidase Inhibition by Mangiophelin ®.

| | |
|---|---|
| | % Hyaluronidase |
| Mangiophelin ® | inhibition |

| Conc. (μg/ml) | Mean | SD |
|---|---|---|
| 25 | 31.24 | 0.03 |
| 50 | 59.11 | 0.02 |
| 100 | 100.01 | 0.03 |

IC50 = 44.34 μg/ml

### Anti Collagenase Activity

Collagen is the most abundant protein structure in the human dermis layer providing the tensile strength of the skin; whereas elastin, a fiber network located in the connective tissue, is responsible for the elastic recoil property. Indeed, collagen and elastin are necessary to the skin which play major role for the healthy and youthful looking skin. However, the accumulated ROS after skin exposure to photoaging stressors can indirectly activate dermal enzymes such as collagenase and elastase which basically break down and degrade collagen as well as elastin, respectively. In essence, the synthesis of elastase and collagenase promotes premature skin [See: Jiratchayamaethasakul et al Invitro Screening of elastase, collagenase, hyaluronidase, and tyrosinase inhibitory and antioxidant activities of 22 halophyte plant extracts for novel cosmeceuticals 23(6) 2020].

Inhibition of collagenase enzyme was estimated by using Enzchek® collagenase, gelatinase assay kit, Invitrogen, Thermo Fischer Scientific, MA, USA, as per the manufacturer's instructions. The assay was performed using Type IV collagenase from Clostridium histolyticum with DQ™ gelatin as a substrate. Different concentrations of Mangiophelin® were pre-incubated for 10 min at room temperature with 20 μl of 12.5 μg/ml gelatin substrate. Hundred microliters of the 0.8 U/ml Collagenase enzyme solution was added, and the fluorescence intensity was measured at Em: 520 nm and Ex: 485 nm after 30 min of incubation using BMG FLUOstar Optima Microplate reader. A dose dependent inhibition of collagenase activity was shown (Table 9, FIG. 4B).

TABLE 9
Collagenase Inhibition by Mangiophelin ®

| | |
|---|---|
| | % Collagenase |
| Mangiophelin ® | enzyme inhibition |

| Conc (μg/ml) | Mean | SD |
|---|---|---|
| 25 | 11.40 | 1.62 |
| 50 | 41.18 | 4.76 |
| 75 | 75.80 | 11.95 |

IC50 = 54.08 μg/ml

### Anti Elastase Activity

Inhibition of elastase enzyme was estimated by using Enzchek® elastase assay kit, Invitrogen, Thermo Fischer Scientific, MA, USA as per the manufacturer's instructions. The assay was performed using DQ elastin from bovine neck ligament as a substrate. A dose dependent inhibition was recorded (Table 10, FIG. 4C).

TABLE 10
Elastase Inhibition by Mangiophelin ®

| | |
|---|---|
| | % Elastase enzyme |
| Mangiophelin ® | inhibition |

| Conc. (μg/ml) | Mean | SD |
|---|---|---|
| 62.5 | 29.69 | 8.06 |
| 125 | 41.45 | 9.57 |
| 250 | 52.02 | 9.41 |
| 500 | 55.70 | 1.85 |
| 1000 | 81.24 | 4.37 |

IC50 = 223.3 μg/ml

### Anti Glycation Activity

Glycation is a non-enzymatic reaction between macromolecules and sugars causing alterations to the elastic fibers and premature aging of the skin.

Different concentrations of Mangiophelin® at a volume of 10 μl were added to 40 μl of 25 mg/ml bovine serum albumin and 50 μl of 150 mg/ml D-Ribose in a 96-well black microplate. D-ribose with buffer served as control. The plate containing the mixture was incubated for 24 h at 37 °C. The advanced glycation end product (AGE) was detected by measuring the fluorescence intensity at Ex/Em of 390/460 nm to evaluate vesperlysines and 320/405 to evaluate pentosidine for AGE formation by using BMG FLUOstar Optima Microplate reader [See: Sero et al. Tuning a 96-well microtiter plate fluorescence-based assay to identify AGE inhibitors in crude plant extracts. Molecules 18(2013), Pgs. 14320-14339]. A dose dependent inhibition of vesperlysines and pentosidine like AGE formation (Table 11, FIG. 4D, FIG. 4E).

TABLE 11
vesperlysines and pentosidine like AGE formation
Inhibition by Mangiophelin ®

| | | |
|---|---|---|
| | % inhibition of | % inhibition of |
| | vesperlysines like | pentosidine like |
| Mangiophelin ® | AGE formation | AGE formation |

| Conc (μg/ml) | Mean | SD | Mean | SD |
|---|---|---|---|---|
| 250 | 20.17 | 2.61 | 16.49 | 0.25 |
| 500 | 32.61 | 4.52 | 18.97 | 7.77 |
| 1000 | 38.32 | 1.19 | 32.98 | 8.53 |

The results indicate that Mangiophelin® is an excellent skin care agent that confers a complete skin protection by reducing signs of skin ageing, rejuvenates the skin by being an antioxidant and lightens the skin and proves an even tone.

### Example 6: Formulation Comprising Mangiophelin®

TABLE 12
Topical Formulations

| Phase | Ingredients | Functions |
|---|---|---|
| A | Purified Water | Diluent |
| | Disodium EDTA | Chelating Agent |
| | Biopol Crystals | Thickener |
| | Glycerin | Humectant |
| B | Olivem 1000 | Emulsifier |
| | DUB CO | Emollient |
| | GMS SE | Emulsifier |
| | Cetyl Alcohol | Emollient |
| | Shea Butter | Emollient |
| | Imex IN3 | Emollient |
| | Tinogard TT | Anti Oxidant |
| | Tinogard TS | Anti Oxidant |
| C | Tween 20 | Solubilizer |
| | Mangiophelin ® | Skin lightening/ |
| | | anti-ageing |
| D | Purified Water | Diluent |
| E | Euxyl PE 9010 | Preservative |
| | Xiameter PMX | Detackification |
| | 3031 | |
| F | Purified Water | Diluent |
| | Sodium | Anti Oxidant |
| | Metabisulfite | |

TABLE 13
Tablet

Active Ingredients
Mangiophelin ®
Excipients
Microcrystalline cellulose

TABLE 14
Capsule

Active Ingredients
Mangiophelin ®
Excipients
Microcrystalline cellulose, Colloidal silicon dioxide, Magnesium
stearate, BioPerine ®, Polyvinylpyrrolidone/starch/Hydroxy
propyl methyl cellulose, Hydroxy propyl cellulose

The above formulation is merely an example, any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid.

2. The composition as in claim 1, wherein the composition is isolated from Mangifera indica fruit using a process comprising:
a) Charging coarse powder of immature deseeded dry fruit in a cleaned reactor;
b) Charging 3 volumes of Ethanol to the same reactor and stir at 60-65 °C for three hours;
c) Filtering the mass from step b, through nutsche filter;
d) Repeating the steps b and c twice;
e) Combining Ethanol filtrates, and concentrating to reduce the volume to ⅛th of the original volume;
f) Washing twice with equal volumes of Ethanol by stirring 1 hr each time at 50 °C and strip off Ethanol (to remove moisture from the concentrate);
g) Dividing the same (Ethanol concentrate) into equal fractions (fraction 1 and fraction 2);
h) Concentrating fraction 1 from step g to pourable mass and dry the product completely at 55-60 °C under high vacuum in vacuum tray dryer (VTD) to yield hygroscopic powder and assayed for polyphenols by UV spectrophotometry;
i) Dissolving fraction 2 from step g with two volumes of de-mineralized water and partition three times with equal volumes of n-Heptane;
j) Partitioning the aqueous phase from step i three times with equal volumes of ethyl acetate;
k) Concentrating the ethyl acetate portion to pourable mass at 60-65 °C under high vacuum;
l) Drying the product completely at 55-60 °C under high vacuum in VTD to yield hygroscopic powder and assayed for polyphenols by UV spectrophotometer;
m) In the left-over aqueous layer from step j, add Maltodextrin (60%) and spray dry;
n) Blending the final product in the ratio of 55.55% initial extract powder (obtained from step h) and 9.02% of enriched extract of step 1 with 35.42% of spray dried material. The product obtained was assayed for polyphenols by UV;
Wherein the polyphenols content is 14% w/w from step h and 60% w/w from step 1 and 12% w/w from step n.

3. The composition as in claim 1, wherein the total polyphenol content is in the range of 10%-60% w/w.

4. The composition as in claim 1, wherein the composition further comprises of stabilizing agents, bioavailability enhancers, antioxidants, cosmeceutically accepted excipients.

5. A method of managing skin ageing in a subject, comprising a) identifying the subject showing signs of skin ageing b) administering effective dose of a standardized polyphenol composition comprising a standardized polyphenol composition from Mangifera indica comprising 1-1.5% w/w Mangiferin, 0.03-0.20% w/w Quercetin, 0.02-0.1% w/w Isoquercetin, 0.18-0.30% w/w Ellagic acid; and 1.42-3.00% w/w Gallic acid, to bring about skin protection in the subject.

6. The method as in claim 5, wherein the composition confers skin protection by reducing signs of skin aging, rejuvenating the skin, reducing allergy, and brightening skin tone.

7. The method as in claim 5, wherein polyphenols are in the range 10-60% w/w.

8. The method as in claim 6, wherein the composition reduces skin aging through collagen enhancement by inhibiting collagenase enzyme, anti-glycation activity, and anti-elastase activity.

9. The method as in claim 6, wherein the composition rejuvenates skin by being an anti-oxidant and inhibiting free radicals.

10. The method as in claim 6, wherein the composition confers anti-allergic activity by inhibiting Histamine and β-hexosaminidase release from mast cells.

11. The method as in claim 6, wherein composition confers skin brightening effect by inhibiting Melanin production and Tyrosinase activity.

12. The method as in claim 6, wherein the signs of skin aging include Wrinkles, Xeroderma, Age spots, Loss of elasticity, Rough skin, Sagging skin, Uneven skin tone, dullness and presence of visible pores and skin tag.

13. The method as in claim 6, wherein the composition further comprises of stabilizing agents, bioavailability enhancers, antioxidants, cosmeceutically or nutraceutically accepted excipients, and administered topically in the form of creams, lotions, ointments, serum, emulsions.

14. The method as in claim 5, wherein the subject is a mammal.
